(19) United States
(12) Patent Application Publication
(10) Pub. No.: US 2024/0122743 A1
(43) Pub. Date: Apr. 18, 2024

(54) CONTRACEPTIVE DEVICE AND ION-RELEASING BODY

(71) Applicant: Meira Dühlmeyer, Bielefeld (DE)

(72) Inventor: Meira Dühlmeyer, Bielefeld (DE)

(21) Appl. No.: 18/277,728

(22) PCT Filed: Dec. 10, 2021
(86) PCT No.: PCT/EP2021/085119

§ 371 Date: Aug. 17, 2023

(30) Foreign Application Priority Data
Feb. 18, 2021 (DE) 102021201558.1
Feb. 18, 2021 (DE) 202021100798.2

(51) Int. Cl. A61F 6/14

(52) U.S. Cl. CPC A61F 6/14

(57) ABSTRACT

The invention relates to a device (10) for contraception, comprising:
at least one thread (5) and
at least one ion-emitting body (3, 3′, 3″, 3′″) having at least one through hole (31) with at least two through hole openings (31c, 31d) through which the at least one thread (5) extends,
wherein an outer surface (32, 32′, 32″, 32′″) of the ion-emitting body (3, 3′, 3″, 3′″) extending from one of the through hole openings (31c, 31d) to the other one of the through hole openings (31d, 31c) has no outwardly facing edge.

The invention relates to a device for contraception and to an ion-emitting body for such a device.

For contraception, intrauterine devices, in short IUD, are known, which enable hormone-free contraception. A frequently used intrauterine device is the so-called classical copper spiral, which consists of a plastic frame, around which copper wire is wound in portions. This is inserted into the womb (uterus) for conception control (contraception). By the release of copper ions, the endometrium and the mucous at the cervix should change and the mobility and life of sperm should be reduced, which ultimately leads to contraception in addition to other active principles discussed. Alternatively, not hormone-free, there are IUD's, which release hormones and exert an anti-conceptive effect via the classical way of suppressing the pituitary.

A further variant of an intrauterine device is the so-called copper chain. This variant of the Cu-IUD (copper spiral) consists of a plastic thread with copper tubes threaded thereon. Depending on the size of the uterus, copper chains with four or six such bodies are usually used. An insertion-side end of the plastic thread of the copper chain has a small knot, which is inserted into the uterine muscle for anchoring the copper chain. The copper chain is based on the same principle of action as the copper spiral, but can adapt better to the uterus due to its comparatively small and flexible design. Copper-chain-IUD have the advantage over conventional copper IUDs that their use is also possible, inter alia, in the case of nullipara, since there is no enhancement of the dysmenorrhoea. In addition, the consequence of hypermenorrhoea rarely occurs, as in the case of a conventional copper-IUD. The Pearl index is significantly lower. Longer lying periods can also be achieved, since no plastic portions are required, which can become brittle and, associated therewith, break after an average of 3 to 4 years. Furthermore, dispensing with a plastic frame entails a lower risk of infection. Due to the lack of tubal irritation by plastic arms, the rate of extrauterine pregnancies can be reduced. According to their flexible construction, copper chains are often also referred to as frameless intrauterine devices.

In spite of the small and flexible configuration of the copper chain, so-called cycle-independent IUP-induced spotting or intermenstrual bleeding could be detected in women who have received it. As a possible cause for this, microerosions at the endometrium via the sharp-edged ends of the copper tubes of the copper chain (i.e. injuries of endometrial vessels) could be detected by hysteroscopy in more than 300 cases as a cause.

In view of the above observation, the object of the present invention is to provide a frameless intrauterine device which can reduce or even prevent erosions at the endometrium.

The object is achieved by a device for contraception according to claim 1 and an ion-emitting body for such a device according to claim 15. Advantageous developments of the invention are provided in the dependent claims.

According to the invention, the device for contraception comprises at least one thread and at least one ion-emitting body having at least one through hole with at least two through hole openings through which the at least one thread extends, wherein an outer surface of the ion-emitting body extending from one of the through hole openings to the other one of the through hole openings has no outwardly facing edge.

The elimination of outwardly facing edges, as would occur, for example, when using copper tubes at the end faces in the transition to the outer side surfaces, minimizes the risk of erosions at the endometrium. In addition, the strongest erosions were to be observed by the edges which are produced by the fastening by means of folding pressing onto the thread.

Even if the ion-emitting body may still have, for example, an edge at the transition of the outer surface to the through hole, this edge does not face outwards, however, but rather merely forms a surface recess by the respective through hole opening. In addition, the transition from the outer surface to the through hole may also be formed via a radius. A decisive advantage over the copper tubes is also the elimination of the sharp-edged ends by the fastening pressing. The present invention offers a total elimination of the sharp-edged ends via the through holes with recessing of the knot fastening into the body interior.

A thread made of polypropylene, for example, may be used as the thread. The term “thread” comprises limp structures having a dominant one-dimensional extent and is directed not only to threads formed from plastic, fibers or other materials, but may also be formed by other cords or chains in a thread-like form. In addition, combinations of a plurality of such threads, for example for reinforcement, may also be used. For simplicity, the term thread is used in the following, although the disclosure in this regard is also equally transferable and thus applicable to several threads, provided that this is not excluded in the context. The ion-emitting bodies may be restricted in their moving ability relative to the thread via knots, the diameter of which is greater than the minimum diameter of the through hole of the respective ion-emitting body, at least in one direction of extent of the thread. One end of the thread has an anchorage knot as a device fixation, which may be inserted into the uterine muscle when the device is inserted into an uterus, in order thereby to hold the device in the uterus. In addition, a metal clip may be placed below the anchorage knot or between the anchorage knot and the at least one ion-emitting body, respectively, in order to form a sonographically detectable reference. For example, a stainless steel clip may be used for this purpose. However, the metal clip may also be formed from another metal or a corresponding alloy, which, in addition or as an alternative to a sonographical detectability, also has a bacterio- and/or fungistatic effect, in order to be able to counteract corresponding influences by inserting the device into the uterine muscle.

In an embodiment, the outer surface of the ion-emitting body is at least in sections describable as a surface section of an ellipsoid with a, b and c as lengths of the semi-axes of such an ellipsoid, in particular in Cartesian coordinates via the equation

$$\frac{{x}^{2}}{{a}^{2}} + \frac{{y}^{2}}{{b}^{2}} + \frac{{z}^{2}}{{c}^{2}} = 1$$

wherein a, b and c are greater than zero.

The outer surface of the ion-emitting body is thus formed at least in sections via surface sections of an ellipsoid. In particular, the basic shape of the ion-emitting body as a whole has substantially the shape of an ellipsoid. The term “substantially” relates to a recognizable basic shape, wherein, depending on the specific embodiment, individual surface regions of the ion-emitting body may be formed recessed and/or protruding in order, for example, to enlarge the surface for the release of ions. For example, the surface may have concave inwardly directed bulges, similar to the surface structure of a golf ball, as recessed surface regions. Analogously to this, there is also the possibility of convex outwardly directed bulges, wherein protruding surface regions according to the basic concept of the invention have no edges or tips which entail the risk of erosions. However, the bulges cited here by way of example do not lead to a change in the recognizable basic shape of the ion-emitting body as an ellipsoid. The recesses in the outer surface of the ion-emitting body formed by the through-bore openings of the through-bore also do not lead to a substantial change in the recognizable basic shape.

If the outer surface of the ion-emitting body in the recognizable basic shape is formed only in sections by surface sections of an ellipsoid, the remaining outer surface is also to be configured here such that the surface transitions are round.

With regard to the description of the outer surface or of surface sections of the outer surface by way of an ellipsoid, the lengths of the semi-axes a, b and c represent the respective lengths in three axes perpendicular to one another. In a Cartesian coordinate system, for example, the length of the semi-axis a corresponds to the length of the semi-axis in the x-direction, the length of the semi-axis b corresponds to the length of the semi-axis in the y-direction and the length of the semi-axis c corresponds to the length of the semi-axis in the z-direction.

In particular, a longitudinal axis of the ion-emitting body extends substantially parallel to an extension direction of the through hole of the ion-emitting body and the lengths of the semi-axes a, b extend perpendicular to the longitudinal axis and the length of the semi-axis c extends in the direction of the longitudinal axis.

Accordingly, the direction of the longitudinal axis of the ion-emitting body corresponds substantially to the extension direction of the through hole and is in particular coaxial thereto. The term “substantially” in relation to the extension direction relates to the fact that the through hole does not necessarily have to be rectilinear, but an extension direction can nevertheless be specified as a connection of the through hole openings on the input side and on the output side.

According to an embodiment, the lengths of the semi-axes a, b and c are equal.

The outer surface of the ion-emitting body thus forms, as a whole or at least in sections, a sphere as a shape of an ellipsoid with semi-axes a, b and c of equal length.

In particular, the lengths of the semi-axes a, b and c are between 1 mm and 5.5 mm, in particular between 1.5 mm and 5 mm.

By means of larger lengths of the semi-axes, larger sphere diameters and thus larger surfaces for ion emission may be provided, specifically with regard to the outer surface of the ion-emitting body as a whole as a sphere. On the other hand, for example, smaller spheres may be used more flexibly as ion-emitting bodies.

Alternatively, two of the lengths of the semi-axes a, b and c are equal and one of the lengths of the semi-axes a, b and c is different therefrom.

The outer surface of the ion-emitting body thus forms a rotational ellipsoid in its basic shape. Alternatively, the outer surface may also have only at least one surface section, which is formed via a corresponding surface section of an ellipsoid.

If the outer surface of the ion-emitting body is formed in its basic shape by a rotational ellipsoid, a larger surface for ion emission may be provided thereby, for example, in comparison to a sphere with the same volume.

In an embodiment, the lengths of the semi-axes a and b are equal.

For example, the length of the semi-axis c extends parallel, in particular coaxial, to the longitudinal axis of the ion-emitting body. The longitudinal axis of the ion-emitting body is substantially parallel to the extension direction of the through hole of the ion-emitting body. Accordingly, with equal lengths of the semi-axes a and b and a different length of the semi-axis c, a rotationally symmetrical ellipsoid results with the longitudinal axis of the ion-emitting body as the rotational axis for the symmetry. A tilting of the ion-emitting body about an axis perpendicular to the longitudinal axis is less probable at least with a coaxial position of the longitudinal axis with respect to the extension direction of the through hole.

In particular, the lengths of the semi-axes a and b are between 1 mm and 2 mm, in particular about 1.5 mm, and the length of the semi-axis c is between 1.5 mm and 5.5 mm, in particular between 2 mm and 5 mm.

For an ion-emitting body as a rotational ellipsoid with its longitudinal axis as the rotational axis, this results in dimensions of 2 mm to 4 mm, in particular 3 mm, for a circle diameter perpendicular to the longitudinal axis, and 3 mm to 11 mm, in particular 4 to 10 mm, for the length in the direction of the longitudinal axis. In particular with a greater length in the direction of the longitudinal axis compared to the circle diameter, with the same surface, the circle diameter may be reduced, in order to facilitate or at least not hinder an insertion of the device for insertion into the uterus.

Alternatively to the embodiment of semi-axes a and b of equal length, the lengths of the semi-axes a and c, or b and c are equal.

The ion-emitting body may thereby form a rotational ellipsoid with a rotational axis perpendicular to the above-described longitudinal axis.

In particular, the length of the semi-axis c is between 1 mm and 5.5 mm, in particular between 1.5 mm and 5 mm, and the length of the semi-axis a or b different therefrom is between 1 mm and 3.5 mm, in particular between 1.5 mm and 3 mm.

Especially with a shorter length of the semi-axis c compared to the length of the semi-axis a or b different therefrom, the ion-emitting body may tilt more easily in the insertion direction during an insertion of the device for insertion into the uterus, so that in spite of a one-sided larger dimension, a hindrance during the insertion process may thereby be avoided.

According to a further embodiment, the lengths of the semi-axes a, b and c are each different from one another.

The ion-emitting body may thereby be formed or described, for example, as a triaxial or three-axis ellipsoid, respectively. Here, as in all other embodiments, however, in which the outer surface of the ion-emitting body is describable in its basic shape by an ellipsoid, comparatively simple manufacturing specifications still result in spite of individual configuration variants, such as with regard to dimensions and/or surface structures.

Advantageously, the through hole has at least two different diameters.

By means of at least two different diameters of the through holes, a larger surface may be provided, for example, in a section of the through hole having the larger diameter, while the ion-emitting body may cooperate with the knot in the thread for restricting the relative movement of the ion-emitting body, at least by means of the section of the through hole having the smaller diameter. If the larger diameter is larger and the smaller diameter is smaller than the knot, the knot may be covered by means of the section of the through hole having the larger diameter, so that the latter does not form an interfering contour or the distance of a plurality of ion-emitting bodies of the device may also be reduced. The through hole may also have more than two sections having different diameters, in order to be able to offer flexible stops, for example with respect to different knot sizes and/or distances to be provided of a plurality of ion-emitting bodies. In view of the above examples, the different diameters are to be designed in particular so that they decrease in one direction, in this case preferably in the direction of the anchoring knot, or increase.

According to an embodiment, the ion-emitting body contains or is formed from a metal, in particular copper, a copper alloy, gold, a gold alloy or a copper-gold alloy.

For example, by copper ions which are released when using copper or an alloy containing copper, a toxic and inhibitory effect on sperm can be brought about, which may lead to a reduction in the mobility and life of the sperm. In addition, the use of gold or a gold-copper alloy or copper-gold alloy, respectively, may have a bacteria- and fungistatic effect, which may reduce the risk of infections and inflammations. Likewise, a microgalvanic effect in the case of a gold-copper alloy or copper-gold alloy, respectively, may have positive effects on the contraception.

The above-mentioned metals and/or their alloys may form the ion-emitting body completely or only partially. For example, also only a base body of the ion-emitting body, which consists of plastic, for example, for cost reasons, may be provided with a coating with one of the above-mentioned metals and/or their alloys. The coating may also be restricted to a surface which is suitable for sufficient ion emission. The coating is thereby exposed to the outside for ion emission.

Advantageously, the device comprises 2 to 5, in particular 3 or 4, ion-emitting bodies through the respective through holes of which the at least one thread extends.

The number of ion-emitting bodies is thereby adapted to the total amount of ions to be emitted as well as dimensions and geometric configurations of the ion-emitting bodies suitable for reception. The ion-emitting bodies through which the thread passes may be the same and/or different in their dimensions, their geometric configurations and/or their material selection. In the case of at least partially different ion-emitting bodies, i.e. at least two ion-emitting bodies which differ in a characteristic feature, these may be assembled flexibly according to the use to be provided.

A further aspect of the invention relates to an ion-emitting body for a device for contraception according to the above description.

Possible configurations and associated advantages of the respective ion-emitting bodies result analogously to the above description.

The invention is explained in more detail below with reference to the accompanying figures. The figures show in detail:

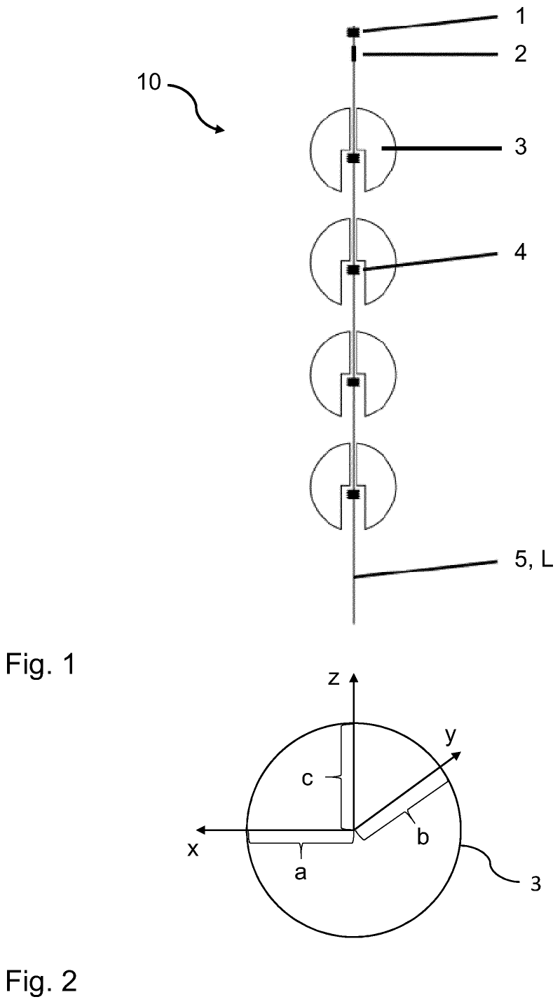

FIG. 1 a schematic cross-sectional view along a longitudinal axis of a device for contraception according to a first embodiment of a device according to the invention;

FIG. 2 a schematic view of an ion-emitting body for illustrating the description as an ellipsoid;

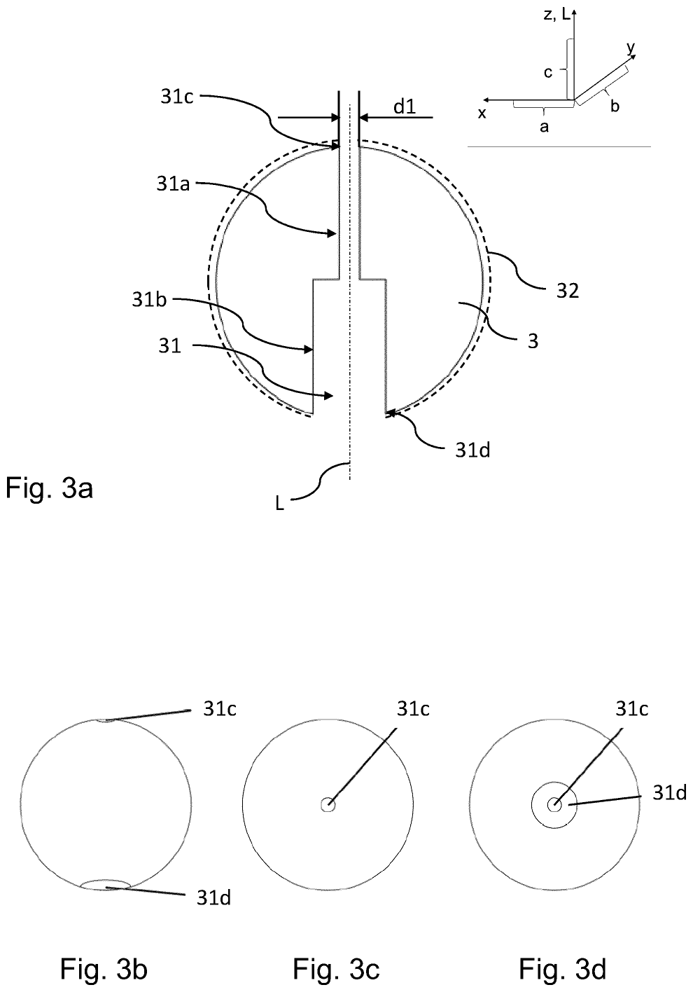

FIG. 3a a schematic cross-sectional view of an exemplary first embodiment of an ion-emitting body along the longitudinal axis of the through hole;

FIG. 3b a side view of the ion-emitting body according to FIG. 3a;

FIG. 3c a view from above of the ion-emitting body according to FIGS. 3a and 3b;

FIG. 3d a view from below of the ion-emitting body according to FIGS. 3a, 3b and 3c;

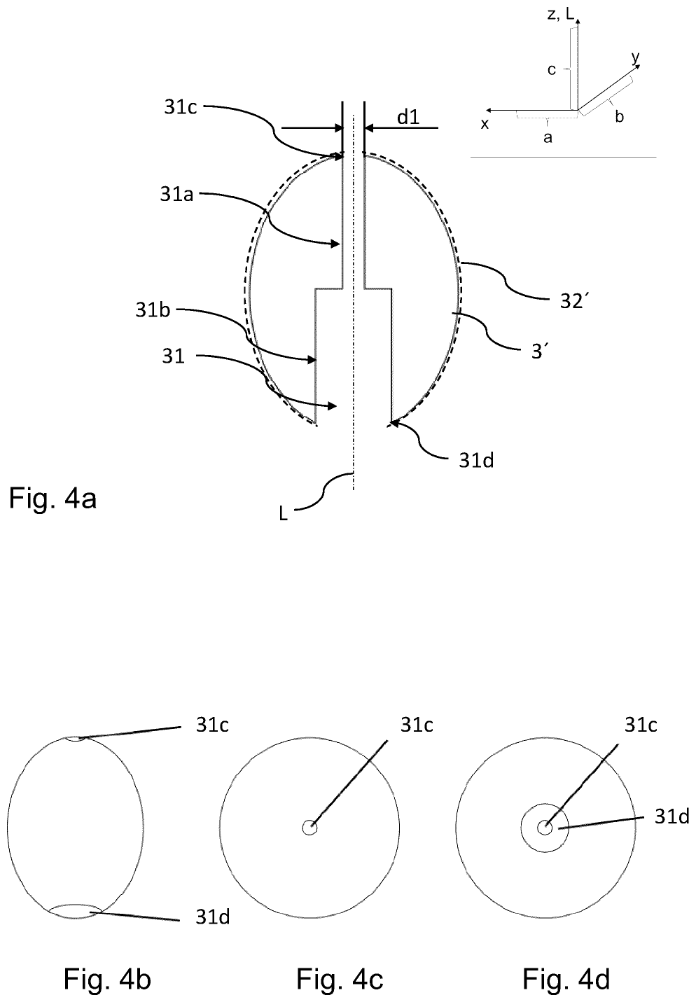

FIG. 4a a schematic cross-sectional view of an exemplary second embodiment of an ion-emitting body along the longitudinal axis of the through hole;

FIG. 4b a side view of the ion-emitting body according to FIG. 4a;

FIG. 4c a view from above of the ion-emitting body according to FIGS. 4a and 4b;

FIG. 4d a view from below of the ion-emitting body according to FIGS. 4a, 4b and 4c;

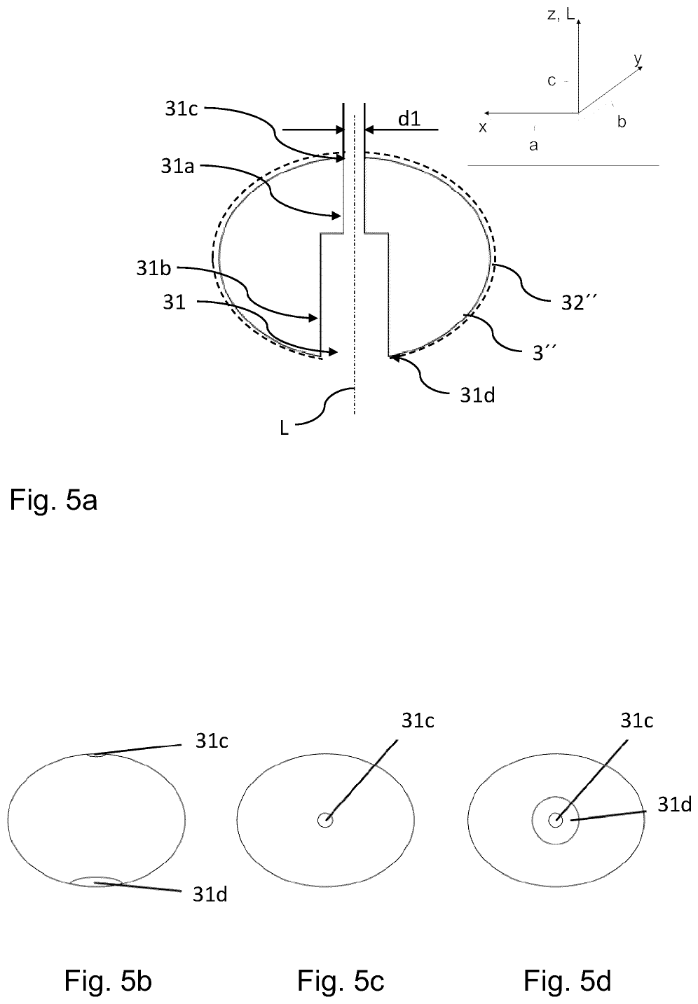

FIG. 5a a schematic cross-sectional view of an exemplary third embodiment of an ion-emitting body along the longitudinal axis of the through hole;

FIG. 5b a side view of the ion-emitting body according to FIG. 5a;

FIG. 5c a view from above of the ion-emitting body according to FIGS. 5a and 5b;

FIG. 5d a view from below of the ion-emitting body according to FIGS. 5a, 5b and 5c;

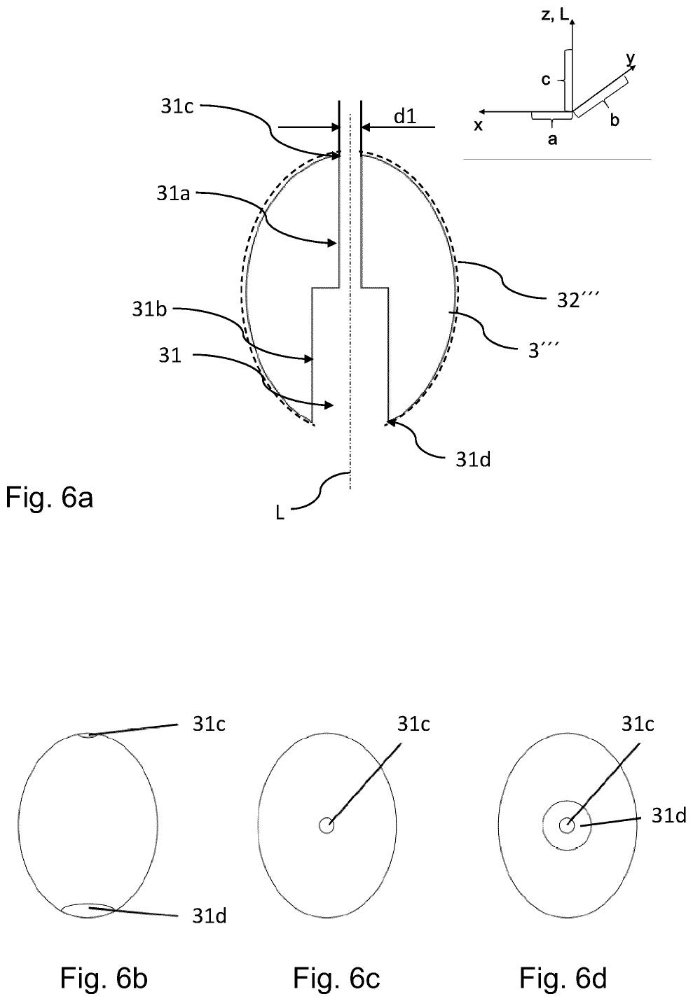

FIG. 6a a schematic cross-sectional view of an exemplary fourth embodiment of an ion-emitting body along the longitudinal axis of the through hole;

FIG. 6b a side view of the ion-emitting body according to FIG. 6a;

FIG. 6c a view from above of the ion-emitting body according to FIGS. 6a and 6b; and

FIG. 6d a view from below of the ion-emitting body according to FIGS. 6a, 6b and 6c.

The terms for the view references, such as “from above”, “from below” or side view, relate to the drawing view according to FIG. 1 or FIGS. 3a, 4a, 5a and 6a, respectively. Figuratively, the term “from above” relates to a region of an ion-emitting body which points in the extension direction of the thread running through it in an insertion direction of a device for contraception. The other view details are derived analogously therefrom.

FIG. 1 shows a schematic cross-sectional view along a longitudinal axis L of a device 10 for contraception according to a first embodiment of a device according to the invention. The device 10 comprises four ion-emitting bodies 3 and a thread 5 which extends through through holes 31 (FIGS. 3a, 4a, 5a, 6a) of the ion-emitting bodies 3. The longitudinal axis L relates to an extension direction of the through holes 31 of the ion-emitting bodies 3 which corresponds here to the extension direction of the thread 5 through the ion-emitting bodies 3. Even if four ion-emitting bodies 3 are shown in the present embodiment, the number of ion-emitting bodies may also differ therefrom. The number of ion-emitting bodies 3 may also be smaller or greater according to the effects to be provided and associated selection of the dimensions, geometric shapes and/or materials.

The thread 5 has four knots 4 which function as spacers of the ion-emitting bodies. For this purpose, the knots 4 are dimensioned such that they are greater than a minimum diameter of the through holes. Accordingly, the ion-emitting bodies 3 may move relative to the thread 5 between two of the knots 4 in each case only at the distance predetermined thereby. As an alternative to the use of the knots 4, however, other thread thickenings or clips arranged on the thread 5 may also be used, the dimensions of which enable a corresponding function. In addition, the thread 5 has an anchorage knot 1 at an insertion-side end for inserting the device 10 into an uterus. The anchorage knot 1 is inserted into the uterine muscle and held there for fixing the device 10 in an uterus. In addition, the anchorage knot 1 is likewise dimensioned such that the ion-emitting body 3 facing the anchorage knot 1 is thereby equally restricted in its relative movement and is thus held on the thread 5. All ion-emitting bodies 3 are thus arranged in each case between two knots 4 or one knot 4 and the anchorage knot 1 and have a predetermined movement clearance as a result. Alternatively, the knots 4 and/or the anchorage knot 1 may also be spaced apart from one another such that at least one of the ion-emitting bodies 3 has no movement clearance and is therefore held in a fixed positional relationship with respect to the thread 5. In the embodiment shown here, the thread 5 additionally has a stainless steel clip 2 which is arranged between the anchorage knot 1 and the ion-emitting body 3 facing the anchorage knot 1. This serves for sonographical detection and may be detected, for example, during the insertion or implantation, respectively, of the device 10 into an uterus as an orientation aid. Accordingly, the stainless steel clip 2 is located in particular in the vicinity of the anchorage knot 1. In a variant, the stainless steel clip 2 may also be dimensioned such that it restricts, instead of the anchorage knot 1, the relative movement of the body 3 facing the anchorage knot 1 in the direction of the anchorage knot 1.

FIG. 2 shows a schematic view of the ion-emitting body 3 for illustrating the description as an ellipsoid. The ion-emitting body 3 according to FIG. 2 is a sphere as a specific variant of an ellipsoid. For reasons of clarity, the through hole 31 is not shown in this view. In addition, the through hole also has no influence on the recognizable basic shape formed by the outer surfaces of the ion-emitting body 3 as an ellipsoid, here specifically as a sphere.

The description of an ellipsoid using Cartesian coordinates is explained by way of example with reference to the ion-emitting body 3 illustrated in FIG. 2. As the ion-emitting body 3 may thereby be described as a sphere, further ellipsoids differing from a sphere or corresponding surface sections of an ion-emitting body may alternatively also be described.

The description of an ellipsoid is made here via the length of its semi-axes a, b and c, the absolute value of which is greater than zero in each case. In Cartesian coordinates, the ellipsoid is then representable via the equation

$$\frac{{x}^{2}}{{a}^{2}} + \frac{{y}^{2}}{{b}^{2}} + \frac{{z}^{2}}{{c}^{2}} = 1 .$$

The length of the semi-axis a corresponds to a length of the semi-axis in the x-direction, the length of the semi-axis b corresponds to a length of the semi-axis in the y-direction and the length of the semi-axis c corresponds to a length of the semi-axis in the z-direction, wherein the direction axes are each perpendicular to one another. In the embodiments shown, the z-direction in each case points in the direction of the longitudinal axis L. In principle, however, the coordinate system may also be oriented differently therefrom. According to the sphere shown in FIG. 2 as basic shape of the ion-emitting body 3, the lengths of the semi-axes a, b and c are equal here.

FIG. 3a shows a schematic cross-sectional view of an exemplary first embodiment of an ion-emitting body 3 along the longitudinal axis L of the through hole 31. As described above, the basic shape of the ion-emitting body 3 in the first embodiment is a sphere. In other words, the outer surface 32 of the ion-emitting body 3 substantially forms a sphere. The spherical shape of the outer surface is interrupted only by through hole openings 31c and 31d of the through hole 31. If such an interruption was intended to be of importance at all with regard to a basic shape, the outer surface may however still be described at least via surface sections of a corresponding ellipsoid.

The longitudinal axis L of the through hole extends centrally through the ion-emitting body 3. With respect to the longitudinal axis L, the length of the semi-axis c is specified here as the length of the semi-axis of the ion-emitting body 3 in the direction of the longitudinal axis L and corresponds to the sphere radius in the sense of equally long semi-axes a, b and c. For orientation, a corresponding coordinate system is also specified again in FIG. 3a, top right. In a variant, the longitudinal axis L of the through hole 31 may however also extend eccentrically through the ion-emitting body 3.

Further, the through hole 31 has two through hole portions 31a and 31b in the direction of the longitudinal axis L with different diameters d1 with respect to the through hole portion 31a and d2 with respect to the through hole portion 31b. The through hole portion 31b extends from the through hole opening 31d in the direction of the longitudinal axis L into the ion-emitting body 3, and the through hole portion 31a extends from an end of the through hole portion 31b facing away from the through hole opening 31d in the direction of the longitudinal axis L up to the through hole opening 31c. The diameter d1 of the through hole portion 31a is smaller than the diameter d2 of the through hole portion 31b and dimensioned such that a knot 4 of the thread 5 cannot pass through the through hole portion 31a. The larger diameter d2 has a diameter which is suitable for reception of a knot 4 of the thread 5. Accordingly, the transition from the through hole portion 31b having the larger diameter d2 to the through hole portion 31a having the smaller diameter d1 forms a stop for a knot 4 of the thread 5, a knot 4 being covered in the stop position by the through hole portion 31b having the larger diameter d2. Here, the diameters d1 and d2 are constant in the direction of the longitudinal axis L. In a variant, however, the through hole portions 31a and 31b may also have variable diameters, the diameter d1 with respect to the through hole portion 31a relating to a minimum diameter and the diameter d2 with respect to the through hole portion 31b relating to a maximum diameter. In a further variant, the through hole 31 may also taper from the through hole opening 31d to the through hole opening 31c at least from a minimum diameter d2 to a maximum diameter d1.

The arrangement of the ion-emitting body 3 with respect to the thread 5 of the device is made such that the through hole opening 31c as an opening of the through hole portion 31a having a smaller diameter d1 is located outwardly on a side of the ion-emitting body 3 facing the anchorage knot 1 and the through hole opening 31d as an opening of the through hole portion 31b having a larger diameter d2 is located outwardly on a side of the ion-emitting body 3 facing away from the anchorage knot 1.

FIGS. 3b, 3c and 3d each again show further external views of the ion-emitting body 3. FIG. 3b shows a side view, FIG. 3c a view from above, i.e. from outside onto the through hole opening 31c, and FIG. 3d a view from below, i.e. from outside onto the through hole opening 31d with the through hole opening 31c located behind it in this viewing direction.

In the embodiment shown, the sphere radius is 1.5 mm, but is not restricted thereto. In variants, the sphere radius may for example also be 2 mm, 2.5 mm, 3 mm, 3.5 mm, 4 mm, 4.5 mm or 10 mm. Further sizes or also intermediate sizes are conceivable.

FIG. 4 a shows a schematic cross-sectional view of an exemplary second embodiment of an ion-emitting body 3′ along the longitudinal axis L of the through hole 31. The second embodiment differs from the first embodiment by the formation of the outer surface 32′ of the ion-emitting body 3′ as a rotational ellipsoid or as a surface section of such a rotational ellipsoid, respectively, if the recesses formed via the through hole 31 are taken into consideration as a deviation, wherein this has no influence on the basic shape.

The ion-emitting body 3′ here has a rotational axis corresponding to the longitudinal axis L with regard to the rotational symmetry of the ion-emitting body 3′ or of the outer surface 32′, respectively. The lengths of the semi-axes a and b as lengths of the semi-axes perpendicular to the rotational axis or longitudinal axis L, respectively, are accordingly equal. In contrast, the length of the semi-axis c in the direction of the longitudinal axis L has a length different from the lengths of the semi-axes a and b. In FIG. 4a, the length of the semi-axis c is longer than the lengths of the semi-axes a and b. According to a variant, however, the length of the semi-axis c may also be shorter than the lengths of the semi-axes a and b.

In the embodiment shown, the length of the semi-axis c is exemplarily 2 mm and the lengths of each of the semi-axes a and b is 1.5 mm. However, the length of the semi-axis c may be, for example, 2.5 mm, 3 mm, 3.5 mm, 4 mm, 4.5 mm or 5 mm and the length of each of the semi-axes a and b may vary likewise.

FIGS. 4b, 4c and 4d also each again show further external views of the ion-emitting body 3′ for the second embodiment. FIG. 4b shows a side view, FIG. 4c a view from above, i.e. from outside onto the through hole opening 31c, and FIG. 4d a view from below, i.e. from outside onto the through hole opening 31d with the through hole opening 31c located behind it in this viewing direction.

FIG. 5 a shows a schematic cross-sectional view of an exemplary third embodiment of an ion-emitting body 3″ along the longitudinal axis L of the through hole 31. The third embodiment differs from the second embodiment in that the rotational ellipsoid is not formed here about the longitudinal axis L as symmetry axis, but about an axis perpendicular to the longitudinal axis L, here the axis x as extension direction of the length of the semi-axis a. In other words, the lengths of the semi-axes b and c are equal here, wherein the length of the semi-axis a differs therefrom and is longer than the length of the semi-axes b and c.

The lengths of the semi-axes b and c are each 1.5 mm here and the length of the semi-axis a is 2 mm. In other variants, other lengths of the semi-axes a, b and c may also be provided here. For example, the length of the semi-axis a may also be 2.5 mm or 3 mm.

FIGS. 5b, 5c and 5d also each again show further external views of the ion-emitting body 3″ for the third embodiment. FIG. 5b shows a side view, FIG. 5c a view from above, i.e. from outside onto the through hole opening 31c, and FIG. 5d a view from below, i.e. from outside onto the through hole opening 31d with the through hole opening 31c located behind it in this viewing direction.

FIG. 6 a shows a schematic cross-sectional view of an exemplary fourth embodiment of an ion-emitting body 3′″ along the longitudinal axis L of the through hole 31. The fourth embodiment differs from the third embodiment in that here the length of the semi-axis a is shortened compared to the equally long semi-axes b and c.

The lengths of the semi-axes b and c are each 2 mm here and the length of the semi-axis a is 1.5 mm. In other variants, other lengths of the semi-axes a, b and c may also be provided here. For example, the lengths of the semi-axes b and d may also be 2.5 mm, 3 mm, 3.5 mm, 4 mm, 4.5 mm or 5 mm.

FIGS. 6b, 6c and 6d also each again show further external views of the ion-emitting body 3″ for the fourth embodiment. FIG. 6b shows a side view, FIG. 6c a view from above, i.e. from outside onto the through hole opening 31c, and FIG. 6d a view from below, i.e. from outside onto the through hole opening 31d with the through hole opening 31c located behind it in this viewing direction.

The ion-emitting bodies 3, 3′, 3″ and 3′″of the first to fourth embodiments are each made of copper. In alternative variants, however, the ion-emitting bodies 3, 3′, 3″ and 3′″ may also be formed from a copper alloy, in particular from a copper-gold alloy, or from a gold or gold alloy, in particular a gold-copper alloy, or be coated at least partially on the outside with a correspondingly aforementioned material.

The invention is not restricted to the described embodiments. Even if only ion-emitting bodies are shown in each case in the above-described embodiments, which, according to their outer surface, reproduce an ellipsoid in their basic shape, the outer surface of the ion-emitting bodies may also have only surface sections of an ellipsoid and otherwise form surface regions which differ therefrom, but which have no protruding edges in transitions. Likewise, the outer surface of the ion-emitting body may be composed of a plurality of surface sections of an ellipsoid. A further variant comprises a surface structuring, for example by concave or convex bulges.

### LIST OF REFERENCE SIGNS

1 Anchorage knot (device fixation)
2 stainless steel clip
3, 3′, 3″, 3′″ ion emitting body
4 knot (spacer)
5 thread
31 through hole
31a through hole portion (d1)
31b through hole portion (d2)
31c through hole opening (d1)
31d through hole opening (d2)
32, 32′, 32″, 32′″ outer surface
a, b, c semi-axes
d1, d2 diameter
L longitudinal axis
x, y, z Cartesian coordinates

1. A device for contraception, comprising:
at least one thread and
at least one ion-emitting body having at least one through hole with at least two through hole openings through which the at least one thread extends,
wherein an outer surface of the ion-emitting body extending from one of the through hole openings to the other one of the through hole openings has no outwardly facing edge.

2. The device for contraception according to claim 1, wherein the outer surface of the ion-emitting body is at least in sections describable as a surface section of an ellipsoid with a, b and c as lengths of the semi-axes of such an ellipsoid, in particular in Cartesian coordinates via the equation
$$\frac{{x}^{2}}{{a}^{2}} + \frac{{y}^{2}}{{b}^{2}} + \frac{{z}^{2}}{{c}^{2}} = 1$$
wherein a, b and c are greater than zero.

3. The device for contraception according to claim 2, wherein a longitudinal axis of the ion-emitting body extends substantially parallel to an extension direction of the through hole of the ion-emitting body, and wherein the lengths of the semi-axes a, b extend perpendicular to the longitudinal axis and the length of the semi-axis c extends in the direction of the longitudinal axis.

4. The device for contraception according to claim 2, wherein the lengths of the semi-axes a, b and c are equal.

5. The device for contraception according to claim 4, wherein the lengths of the semi-axes a, b and c are between 1 mm and 5.5 mm.

6. The device for contraception according to claim 2, wherein two of the lengths of the semi-axes a, b and c are equal and one of the lengths of the semi-axes a, b and c is different therefrom.

7. The device for contraception according to claim 6, wherein the lengths of the semi-axes a and b are equal.

8. The device for contraception according to claim 7, wherein the lengths of the semi-axes a and b are between 1 mm and 2 mm, and the length of the semi-axis c is between 1.5 mm and 5.5 mm.

9. The device for contraception according to claim 6, wherein the lengths of the semi-axes a and c, or b and c are equal.

10. The device for contraception according to claim 9, wherein the length of the semi-axis c is between 1 mm and 5.5 mm, and the length of the semi-axis a or b different therefrom is between 1 mm and 3.5 mm.

11. The device for contraception according to claim 2, wherein the lengths of the semi-axes a, b and c are each different from one another.

12. The device according to claim 1, wherein the through hole has at least two different diameters.

13. The device for contraception according to claim 1, wherein the ion-emitting body contains or is formed from a metal, in particular copper, a copper alloy, gold, a gold alloy or a copper-gold alloy.

14. The device for contraception according to claim 1, wherein the device comprises 2 to 5, ion-emitting bodies through the respective through holes of which the at least one thread extends.

15. An ion-emitting body for a device for contraception according to claim 1.

16. The device for contraception according to claim 5, wherein the lengths of the semi-axes a, b and c are between 1.5 mm and 5 mm.

17. The device for contraception according to claim 8, wherein the lengths of the semi-axes a and b are approximately 1.5 mm.

18. The device for contraception according to claim 8, wherein the length of the semi-axis c is between 2 mm and 5 mm.

19. The device for contraception according to claim 10, wherein the length of the semi-axis c is between 1.5 mm and 5 mm.

20. The device for contraception according to claim 10, wherein the length of the semi-axis a or b different from the length of the semi-axis c is between 1.5 mm and 3 mm.

21. The device for contraception according to claim 14, wherein the device comprises 3 or 4 ion-emitting bodies through the respective through holes of which the at least one thread extends.
